(19) United States
(12) Patent Application Publication
(10) Pub. No.: US 2012/0022569 A1
(43) Pub. Date: Jan. 26, 2012

(54) DEVICE FOR GRADUAL DILATION AND CURETTAGE

(76) Inventor: Mor Bustan, Afula (IL)

(21) Appl. No.: 12/839,424

(22) Filed: Jul. 20, 2010

(51) Int. Cl. A61M 29/02

(52) U.S. Cl. 606/191

(57) ABSTRACT

The subject matter discloses an apparatus for gradually dilating the cervix, comprising an elongated body having sidewalls, at least a portion of said elongated body is located in the cervix and an enlargement mechanism located within the elongated body being in communication with the sidewalls, for generating a movement of the sidewalls away from the central axis of the elongated body, The apparatus further comprises a generation unit located outside the elongated body, for maneuvering the enlargement mechanism such that the movement of the sidewalls is generated.

### BACKGROUND OF THE INVENTION

1. Technical Field

The present disclosure relates to gynecology in general, and to dilation and curettage in particular.

2. Discussion of the Related Art

Dilation, or dilatation, and curettage, literally refer to the dilation (opening) of the cervix and surgical removal of the contents of the uterus. It is a therapeutic gynecological procedure as well as a rarely used method of first trimester abortion. It is commonly referred to as a D&C. Some sources use the term D&C to refer more generally to any procedure that involves the processes of dilation and removal of uterine contents, which includes the more common suction curettage procedures of manual and electric vacuum aspiration. D&Cs are commonly performed to resolve abnormal uterine bleeding, to remove the excess uterine lining in women who have conditions such as polycystic ovary syndrome, to remove tissue in the uterus that may be causing abnormal vaginal bleeding, to remove retained tissue as a method of abortion. D&C is also used in hysteroscopy.

The related art further contains other treatments that require dilation of the cervix, for example Dilation and evacuation or any other treatment desired by a person skilled in the art.

Known methods for dilation of the cervix require a set of vaginal dilators. Such dilators may have a conic or cylindrical shape. Each of the dilators of the set is likely to have another radius to introduce each dilator according to the size of dilation of the cervix. For example, a common set of vaginal dilators contains 8 dilators having radiuses of 2 millimeters (mm), 3 mm, 5 mm, 8 mm, 10 mm, 12 mm, 14 mm and 16 mm. Such set of dilators is used when the required cervix dilation is of about 16-17 mm.

Use of such set requires a skillful medic to know how to introduce each of the dilators. Further, introducing a set of dilators may insert infections to the uterus or the cervix. Further, movement of the dilations performed by the medic may accidentally cause perforation of the cervix or the uterus. It is also inconvenience for the patient to lie while different metallic objects are introduced to such sensitive organs.

It is thus required to provide a safe device and method for cervix dilation without introducing different objects into the uterus and cervix.

### SUMMARY

It is an object of the subject matter to disclose an apparatus for gradually dilating the cervix, comprising:

an elongated body having sidewalls, at least a portion of said elongated body is located in the cervix;

an enlargement mechanism located within the elongated body being in communication with the sidewalls, for generating a movement of the sidewalls away from the central axis of the elongated body;

a generation unit located outside the elongated body, for maneuvering the enlargement mechanism such that the movement of the sidewalls is generated.

The apparatus of claim 1, wherein the enlargement mechanism is a container injected with material by a pump.

The apparatus of claim 2, wherein the material is gas.

The apparatus of claim 2, wherein the pump is a syringe.

The apparatus of claim 1, wherein the elongated body is of a conic shape and the enlargement mechanism is an object inserted into a hollow portion within the elongated body.

### BRIEF DESCRIPTION OF THE DRAWINGS

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are designated by the same numerals or letters.

FIG. 1 schematically illustrates a feminine genital system;

FIGS. 2A and 2B show a cross section of an elongated apparatus inserted into the cervix for gradual dilation thereof;

FIG. 3 shows an apparatus for enlarging the cervix using a pump, according to some exemplary embodiments of the disclosed subject matter;

FIG. 4 shows an apparatus for enlarging the cervix using an object gradually inserted into a hollow volume in the apparatus, according to some exemplary embodiments of the disclosed subject matter;

FIG. 5 shows a cross section of a mechanism for pushing the sidewalls of an apparatus for dilation of the cervix, according to some exemplary embodiments of the disclosed subject matter

### DETAILED DESCRIPTION OF THE PREFERRED EMBODIMENT

The technical problem dealt with in the disclosed subject matter is to dilation of the service without repeatedly inserting objects and removing them from the uterus or from the cervix.

One technical solution suggested in the subject matter is an elongated apparatus comprising sidewalls that can expand their size, such that the radius of the elongated apparatus is enlarged. The elongated apparatus may be of a generally cylindrical shape. A pump that enlarges a container within the inner layer of the elongated apparatus may perform the enlargement. Enlargement of the container results in pushing the sidewalls away from the central axis of the apparatus to the side walls of the cervix and dilate the cervix. Other methods for enlarging the radius of the apparatus may be inserting an object into a hollow volume within the elongated apparatus shaped as a cone, and gradually moving the object into the narrow portion of the cone, such that the object pushes the sidewalls externally and gradually dilate the cervix. Other methods for gradually enlarging the cervix are disclosed below. The apparatus of the disclosed subject matter may be of disposable materials, such as plastics, or made of sterilized materials, such as metal, for example stainless steel.

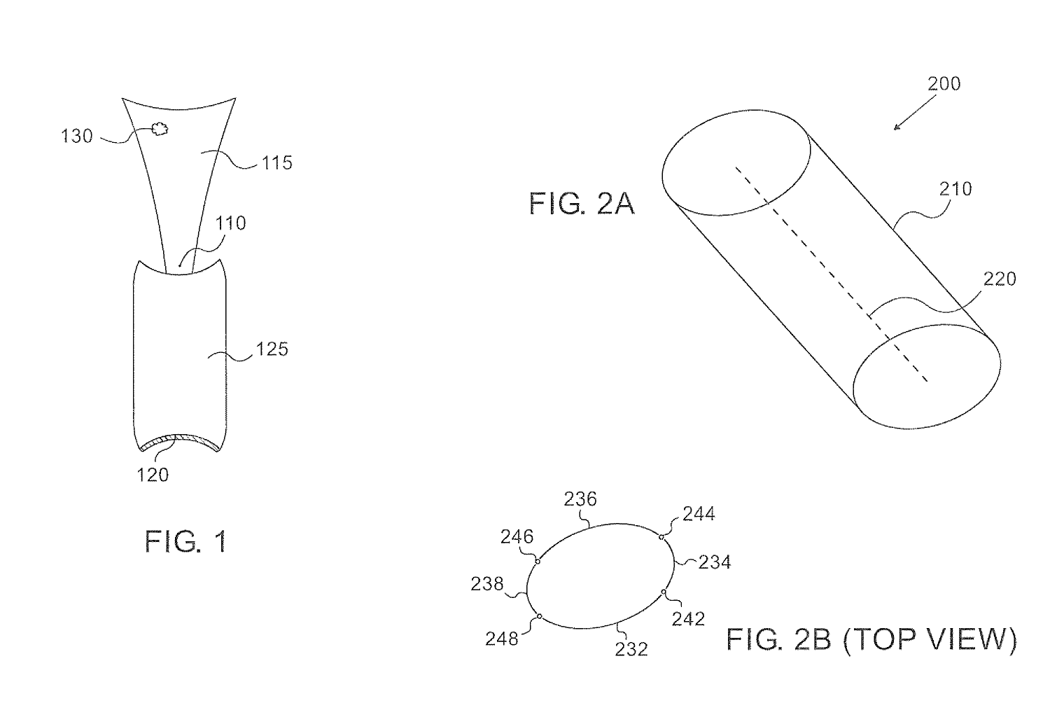

FIG. 1 schematically illustrates a feminine genital system. The feminine genital system comprises a uterus 110, a vagina 120 and a cervix 115. When a medical procedure requires removal of a tissue from the uterus or general examination of the uterus, it is sometimes required to enlarge the cervix in order to remove the tissue if the size of the tissue is larger than the normal opening of the cervix, about 2-3 millimeters. Gradual extension of the cervix, as suggested in the disclosed subject matter, enables a safe, faster and less painful procedure.

FIGS. 2A and 2B show a cross section of an elongated apparatus 200 inserted into the cervix for gradual dilation thereof. The elongated apparatus 200 may be cylindrical, polygonal or a combination of both. The elongated apparatus comprises sidewalls 210 to be extended to the external direction, away from the central axis 220 of the elongated apparatus 200. for enlarging and dilating the cervix. The sidewalls 210 may be assembled of elastic materials such as rubber, plastics, or another material desired by a person skilled in the art. Alternatively, as shown in FIG. 2B, the sidewalls 210 may be assembled of two or more pieces 232, 234, 236, 238 of rigid material such as metal, for example aluminum, connected by elastic elements 242, 244, 246, 248, such as springs or rubber. As such, activating the enlarging mechanism, such as a pump, results in pushing the sidewalls 210 towards the circumference of the elongated apparatus 200, away from the central elongated axis 230 of the elongated apparatus 200. In such case, the two or more pieces 232, 234, 236, 238 of rigid material are pushed away from the central elongated axis 220 and the elastic material is stretched.

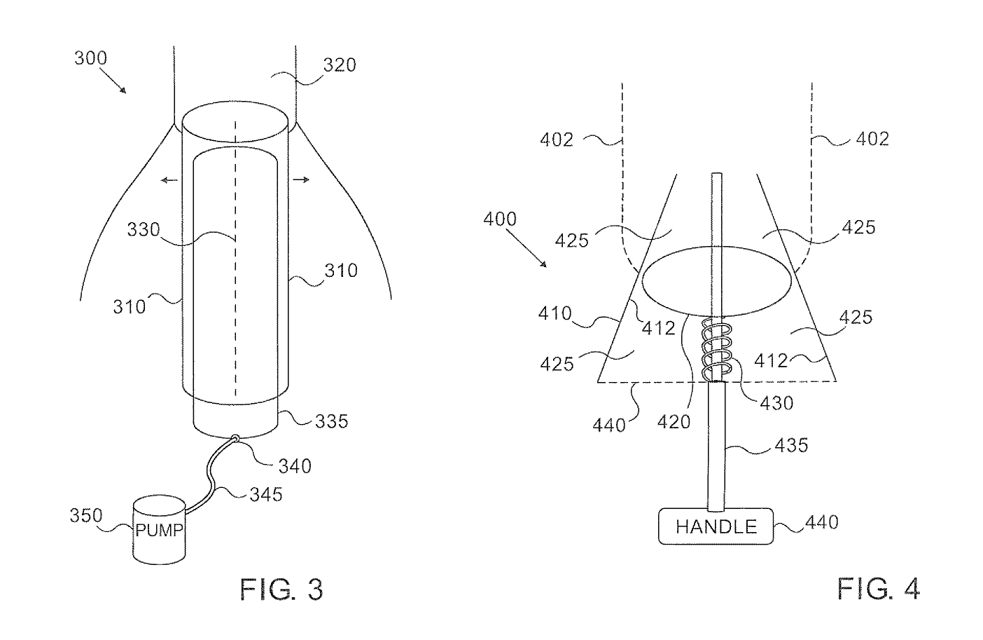

FIG. 3 shows an apparatus for enlarging the cervix using a pump, according to some exemplary embodiments of the disclosed subject matter. The apparatus 300 is of an elongated shape. At least a portion of the apparatus 300 is inserted to the cervix 320. The apparatus 300 comprises sidewalls 310 to be pushed and expand the cervix 320, an activation mechanism and an inner layer to push the sidewalls 310 away from the central axis 330 of the apparatus 300. The inner layer of the embodiment in FIG. 3 is a container 335 inflated by an inflation unit via an aperture 340. A pump 350 inflates the container 335 by pumping material, such as air or fluid into the container 335 via an aperture 340. In some cases, the material is transferred via a tube 345 between the pump 350 to the container 335. The container 335 may be made of an elastic material, such as a sealed nylon bag. A person skilled in the art may desire other materials used for the expandable container 335.

A person, such as a medic, who inflates the container 335, may operate the pump 350. Such operation may comprise squeezing air into the pump 350. Alternatively, the pump 350 may be used as a syringe, gradually injecting material into the container 335. In other embodiments, the pump is used mechanically or electronically, such that the amount of air or fluid or other material injected into the air bag is controlled by the operator of the apparatus 300 or by a control unit (not shown). In other cases, a sensing unit (not shown) is used to detect the size of the apparatus 300, especially the distance between the sidewalls 310 and the central axis 330, and inject material accordingly. The expansion of the container 335 is performed gradually, thus enlarging the cervix gradually. In some exemplary embodiments of the disclosed subject matter, the pump 350 injects 100 milliliter of material every minute to enlarge the cervix 320 by 2 millimeters in every minute.

FIG. 4 shows an apparatus for enlarging the cervix using an object gradually inserted into a hollow volume in the apparatus, according to some exemplary embodiments of the disclosed subject matter. The apparatus 400 comprises sidewalls 410 movable towards the circumference of the apparatus 400 by an enlargement mechanism 420. According to the exemplary embodiment of FIG. 4, the enlargement mechanism 420 is inserted into a hollow volume 425 within the apparatus 400, between the sidewalls 410. The apparatus 400 comprises an inlet 440 through which the enlargement mechanism 420 is inserted into the hollow volume 425. The apparatus 400 may be elliptical or polygonal, and is generally conic, as the distance between the sidewalls and the central longitudinal axis of the apparatus 400 decreases as moving towards the cervix 402. When inserting the enlargement mechanism 420 into the hollow volume 425, the enlargement mechanism 420 touches the inner portion 412 of the sidewalls. A user, such as a medic can maneuver the enlargement mechanism 420 using a handle 440 and a rod 435. The movement of the enlargement mechanism 420 may be linear, directed in the axis between the inlet 440 to the cervix 402. Alternatively, the movement of the enlargement mechanism 420 may be circular, by screwing the enlargement mechanism 420 on a screw thread 430. As such, for example, every round of the enlargement mechanism 420 on the screw thread 430 provides for enlargement of the cervix 402 by 12 millimeters. A sensing unit (not shown may detect enlargement of the cervix 402 by detecting the length of penetration of the enlargement mechanism 420 into the apparatus 400. Alternatively, the sensing unit (not shown) may detect the distance between the sidewalls 410, or the angle between the sidewalls 410 that increases as the enlargement mechanism 420 introduces the hollow volume 425,

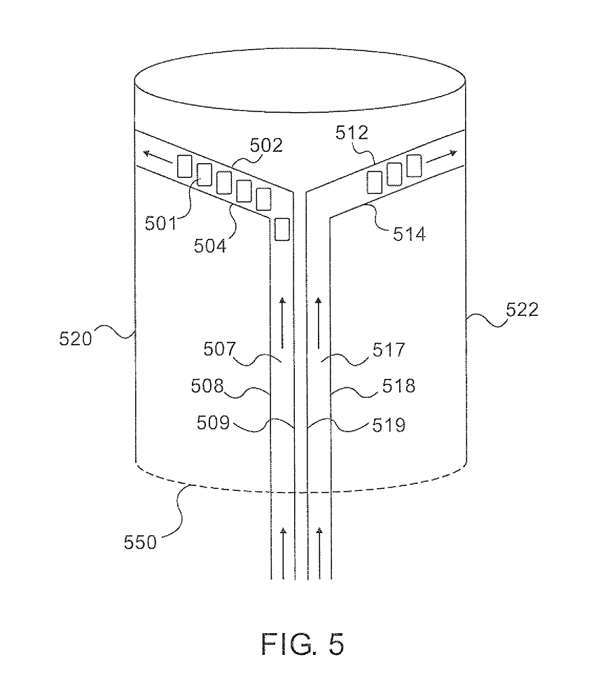

FIG. 5 shows a cross section of a mechanism for pushing the sidewalls of an apparatus for dilation of the cervix, according to some exemplary embodiments of the disclosed subject matter. The apparatus 500 for dilation of the cervix comprises sidewalls 520, 522 pushed by a mechanism operated from outside the apparatus 500. The mechanism comprises one or more tubes, such as tubes 507, 517, within which a rigid material is maneuvered from outside the apparatus 500 to push the sidewalls 520, 522 away from the center of the apparatus 500. The one or more tubes consist of two main segments, the first segment of each tube is generally parallel to the central axis of the apparatus 500, and the second segment is perpendicular to the sidewalls, to be able to push the sidewalls. Tube 507 is limited by borderlines 508 and 509 and tube 517 is limited by borderlines 518 and 519. The tubes may be cylindrical or polygonal, as long as the material such as 501 may flow via the first segment and the second segment. The material is maneuvered by the user or by a control unit connected to a power device to generate movement of the material.

The material may be a plurality or a sequence of metal or rigid pieces as long as a portion of the pieces may move from the first segment to the second segment. The first segment of tube 507 is limited by borderlines 508, 509 and the second segment of tube 507 is limited by borderlines 502, 504. The first segment of tube 517 is limited by borderlines 518, 519 and the second segment of tube 517 is limited by borderlines 512, 514. In some exemplary embodiments of the disclosed subject matter, the material is soft, such as a mixture of clay, or another material that changes its shape upon external pressure. There may be one or more tubes for each of the segments of the sidewalls 520, 522.

The apparatus may comprise an activation element such as a button or switch. The activation may be performed outside the woman's body.

While the disclosure has been described with reference to exemplary embodiments, it will be understood by those skilled in the art that various changes may be made and equivalents may be substituted for elements thereof without departing from the scope of the invention. in addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. An apparatus for gradually dilating the cervix, comprising:
an elongated body having sidewalls, at least a portion of said elongated body is located in the cervix;
an enlargement mechanism located within the elongated body being in communication with the sidewalls, for generating a movement of the sidewalls away from the central axis of the elongated body;
a generation unit located outside the elongated body, for maneuvering the enlargement mechanism such that the movement of the sidewalls is generated.

2. The apparatus of claim 1, wherein the enlargement mechanism is a container injected with material by a pump.

3. The apparatus of claim 2, wherein the material is gas.

4. The apparatus of claim 2, wherein the pump is a syringe.

5. The apparatus of claim 1, wherein the elongated body is of a conic shape and the enlargement mechanism is an object inserted into a hollow portion within the elongated body.
